(19) United States
(12) Patent Application Publication
(10) Pub. No.: US 2019/0029338 A1
(43) Pub. Date: Jan. 31, 2019

(54) FOOTWEAR, IN PARTICULAR SOCKS

(71) Applicant: LENZ GES.M.B.H., Schwarzach (AT)

(72) Inventors: Martin SCHABERREITER, Waldbach (AT); Stefan LENZ, Schwarzach (AT); Gerhard KREMER, Fürstenfeld (AT)

(21) Appl. No.: 16/074,663

(22) PCT Filed: May 15, 2017
(86) PCT No.: PCT/EP2017/061555

§ 371 Date: Aug. 1, 2018

(30) Foreign Application Priority Data
May 19, 2016 (AT) A 253/2016

(51) Int. Cl. A41D 13/005; H05B 3/34; A41B 11/00

(52) U.S. Cl. CPC A41D 13/0051; H05B 3/342; A61F 2007/0045; H05B 2203/036; A41B 11/005

(57) ABSTRACT

Footwear, in particular a sock, includes a leg, with an upper end for receiving a lower leg, a foot part arranged on the leg and having an upper and a lower for receiving a foot, and a toe flap arranged at the foot part, for receiving the toes. The footwear includes at least one heating element with connections for a voltage source, the heating element being provided in the area of the foot part on the upper of the foot part.

The invention relates to footwear, in particular socks, comprising a shank with one shank end for accommodating a lower leg, a foot part located in the shank with a top and a bottom for accommodating a foot, and a toe flap located on the foot part for accommodating toes, the footwear having at least one heating element with terminals for a voltage source.

Moreover, the invention relates to a method for producing footwear.

Generic footwear, in particular a sock or stocking, is known from EP 2 481 309 A1 and AT 505 067 A1.

In previously-known socks or stockings with an integrated heating element, the heating element in the position of use runs underneath the foot. In order to be able to attach the heating element in this position in the sock, the sock, when it comes from the knitting machine, is open above the toes. In the open state of the sock, the heating element is then sewn in, and the opening located above the toes is closed by a seam. Such a heating element has a heating zone, in which a heating wire is generally helically arranged. Here, it is especially disadvantageous that when using such a sock, the wearer is standing on the heating element. This thus reduces wearing comfort and increases the risk of blister formation in corresponding regions of the sole of the foot or the heel due to pressure and friction. Moreover, the risk that the heating element will be damaged, e.g., by breaking an electrical lead, increases; this leads to complete loss of function of the heating element and to complaints.

The object of the invention is to make available footwear with an integrated heating element and a method for producing such footwear, with which the defects of the prior art are avoided.

This object is achieved according to the invention with footwear that has the features of Claim 1 and with a method for producing such footwear that has the features of Claim 12.

Preferred and advantageous embodiments of the invention are the subject matter of the dependent claims.

According to the invention, it is provided that the footwear has at least one heating element with terminals for a voltage source, the heating element being located in the region of the foot part on the top of the foot part. Thus, the wearing comfort can be increased, and the risk of damage and a resulting loss of function of the heating element can be reduced, as a result of which the number of complaints that can be expected is in turn reduced.

In one preferred embodiment, it is provided that the heating element has at least one carrier layer that is preferably made elastic and that can be stretched or its length changed in one direction, and that on or in the carrier layer, there is at least one electrical lead. The electrical lead is preferably located in an undulatory manner at least in zones on or in the carrier layer. Due to the undulatory arrangement of the lead, the inherently inelastic electrical lead together with the carrier layer can be subjected to a change of length (stretched or compressed), without being damaged thereby.

In particular, within the scope of the invention, one embodiment is preferred in which the electrical lead of the heating element is connected to the carrier layer. In this case, the lead can be stitched and/or cemented on the carrier layer. It is especially preferred if the lead has been woven and/or stitched on one surface and/or into the carrier layer, so that a foot held in the footwear comes into contact only with the carrier layer and not with the stitched seams or cement sites.

In one advantageous embodiment, it is provided that the heating element consists of a supply lead and a heating zone, the electrical lead of the heating element in the zone of the supply lead having a preferably lower ohmic resistance than in the area of the heating zone. For this reason, when an electric voltage source is connected to the electrical lead in the region of the heating zone, the electrical lead acts as a heating wire and converts the applied electrical energy at least in part into thermal energy.

In particular within the scope of the invention, one embodiment is preferred in which the heating element is located on the inside of the footwear, in particular sewn on or cemented on. Preferably, the electrical lead is located between the carrier layer and the footwear, so that the lead does not come directly into contact with a foot that is being held in the footwear. An embodiment in which the heating element is located on the outside of the footwear is likewise conceivable.

It is especially preferred if the heating element, in particular the heating zone of the heating element that has at least one heating wire, is located in the toe flap so that the toes of a foot that is being held in the footwear are surrounded at least in part or even entirely by the heating element or the heating zone. For this purpose, the heating element or the heating zone can be made wider or narrower in the toe flap and/or the electrical lead can be located in this region on or in the carrier layer more tightly and/or running differently, for example circularly or in a zigzag pattern.

In one advantageous embodiment, the footwear has a seam, in particular a linking seam, for closing the footwear between the toe flap and bottom of the foot part. Other types of seams, such as, for example, a Rosso seam, or other possibilities for closing the footwear, such as, for example, cementing, are likewise conceivable.

Preferably, the carrier layer of the heating element at least in zones, in particular in the zone of the terminals and in the zone of the transition from the supply lead to the heating zone, has stiffeners that oppose a possible change in length of the carrier layer. In this way, connections located in these zones, such as, for example, soldered or clamp connections, will be protected against mechanical stress, in particular against stretching or compression. The stiffeners are preferably formed by the carrier layer being impregnated in corresponding regions in a medium that is liquid for the time being, such as, for example, textile cement, which subsequently cures and thus stiffens the carrier layer in the indicated regions. Within the scope of the invention, it would also be conceivable to protect these regions against mechanical stress, in particular against stretching, by other precautions, such as, for example, special seams or additional textile layers.

In one preferred embodiment, the heating element is located from the toe flap over the top of the foot part and then along the shank, in particular laterally along the shank as far as to one shank end that is facing away from the foot part. Embodiments in which the heating element is located on the shank in the front or in the back and/or the heating element does not extend over the entire length of the shank or not beyond the foot part or only along the foot part are likewise conceivable.

It can be provided within the scope of the invention that the terminals for the voltage source be located in the region of the shank end, in particular in a cuff zone. In this way, the footwear can be turned up with the cuff zone over the voltage source that has, for example, one or more joined batteries in order to protect, cover up or fix the voltage source in this region.

In another embodiment, the footwear is made reinforced at least in zones, in particular on the shank and/or on the bottom of the foot part, for example by there being in zones one or more additional textile layers on the foot part or by the footwear being fabricated differently or with altered adjustments in zones.

According to the invention, there is furthermore a method for producing footwear according to the invention, in particular a sock, in which the heating element is located in the region of the foot part on the top of the foot part.

It is preferred if the footwear from the shank end to the toe flap is produced, in particular knitted, in the form of a tube, so that an opening remains between the bottom of the foot part and the toe flap. It would also be conceivable, however, for the opening to remain between the top of the foot part and the toe flap or on the end of the toe flap, and/or for the footwear to be manufactured from the toe flap as far as the shank end and/or for the footwear to be manufactured not in the form of a tube, but rather in some other way and/or not in one piece, but from several individual parts.

Preferably, the heating element is subsequently located on the inside or on the outside of the footwear, in particular along the top of the foot part, in particular sewn on or cemented on, for which the footwear can be turned up.

In another preferred method step, the heating element, in particular the heating zone, is located in the toe flap, in particular sewn on or cemented on. Here, it is preferred if essentially the entire inner surface of the toe flap is covered by the heating element. It is just as conceivable, however, for the heating element to cover the inside surface of the toe flap only in zones, preferably the zones that are located above and below the toes of a foot.

Preferably, the heating element is located, in particular sewn on or cemented on, from the toe flap over the top of the foot part along the shank, in particular laterally along it, as far as the shank end. An arrangement of the heating element in reverse sequence is also conceivable, however.

Preferably, the opening that remains after manufacture of the footwear in tube form, in particular between the bottom of the footwear and the toe flap, is closed by a seam, in particular a linking seam. To close this opening, however, other seams or methods can also be used.

The heating element can be provided in zones with stiffeners or other reinforcements against mechanical loads, and/or the footwear can be reinforced at least in zones by applying additional textile layers or by using other or altered manufacturing methods.

Likewise, preferably in the cuff zone, there can be terminals for the electrical lead on or into the footwear, for example as button terminals and/or as clamp or plug-in connections.

Other details, features and advantages of the invention will become apparent from the following description with reference to the attached drawings, in which preferred embodiments of the invention are shown. Here:

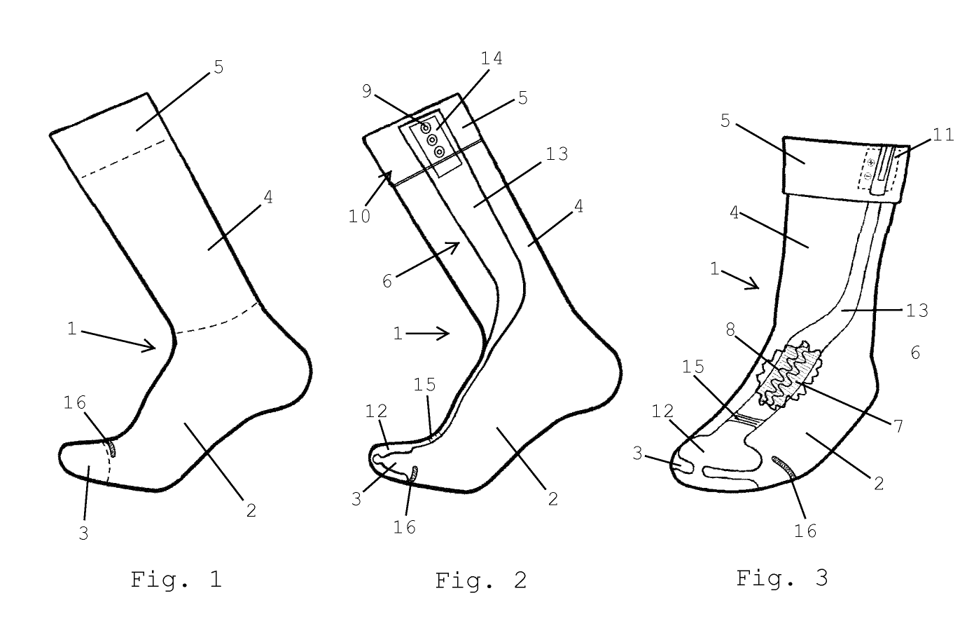

FIG. 1 shows conventional footwear in a side view,

FIG. 2 shows footwear according to the invention in a side view, and

FIG. 3 shows footwear according to the invention in an oblique view with clearance in the region of a foot part.

FIG. 1 shows conventional footwear 1, in particular a sock or a stocking. The footwear 1 has a foot part 2, on one end of the foot part 2 there being a toe flap 3 and on the other end of the foot part 2 a shank 4 with a shank end 5. A seam, in particular a linking seam 16, closes an opening between the top of the foot part 2 and the toe flap 3, which opening forms when the footwear 1 is being manufactured. If conventional footwear 1 has a heating element 6, the latter is located on the bottom of the foot part 2. The bottom of the foot part 2 in the position in which the footwear 1 is being used is located on the sole of the foot.

FIGS. 2 and 3 show footwear 1 according to the invention that, like conventional footwear 1, has a foot part 2, a toe flap 3, a shank 4 and a shank end 5 with a cuff zone 10.

A heating element 6 is located inside on the footwear 1 and has a carrier layer 7 into which an electrical lead 8 is stitched in an undulatory manner. The heating element 6 runs from the shank end 5 laterally along the shank 4 over the top of the foot part 2 as far as the toe flap 3, the electrical lead 8 being located between the carrier layer 7 of the heating element 6 and the footwear 1. On the shank end 5 in the cuff zone 10, the heating element 6 has terminals 9 for a voltage source 11, which are located outside on the footwear 1 on the shank end 5 when the cuff zone 5 is not turned down.

The electrical lead 8 forms a supply lead 13 to one heating zone 12 of the heating element 6, in the heating zone 12 there being at least one heating wire. The heating element 6 furthermore comprises a zone 14, in which the terminals 9 are located, and a transition 15 between the supply lead 13 and the heating zone 12, in which there is a connection between the electrical lead 8 and the heating wire. The heating zone 12 of the heating element 6 is located inside on the toe flap 3 so that toes of a foot held in the footwear 1 are surrounded by the heating element 6.

The zone 14 of the terminals 9 and the transition 15 from the supply lead 13 to the heating zone 12 are made reinforced in order to protect connections located therein from mechanical stresses, such as, for example, from stretching, between the electrical lead 8 and terminals 9 and the electrical lead 8 of the heating zone 12 and the electrical lead 8 of the supply lead 13.

FIG. 3 shows the footwear 1 with the shank end 5 turned down, the voltage source 11 connected to the terminals 9 being located between the shank end 5 turned down to the outside and the shank 4.

The footwear 1 according to the invention, unlike the conventional footwear 1, is closed by a linking seam 16 between the bottom of the foot part 2 and the toe flap 3 and not between the top of the foot part 2 and the toe flap 3.

In summary, one embodiment of the invention can be described as follows:

Footwear 1, in particular a sock, comprises a shank 4 with one shank end 5 for accommodating a lower leg, a foot part 2 located on the shank 4 with a top and a bottom for accommodating a foot, and a toe flap 3 located on the foot part 2 for accommodating toes. The footwear 1 has at least one heating element 6 with terminals 9 for a voltage source 11, the heating element 6 being located in the zone of the foot part 2 on the top of the foot part 2.

1. Footwear (1), in particular a sock, comprising a shank (4) with one shank end (5) for accommodating a lower leg, a foot part (2) located on the shank (4) with a top and a bottom for accommodating a foot, and a toe flap (3) located on the foot part (2) for accommodating toes, the footwear (1) having at least one heating element (6) with terminals (9) for a voltage source (11), wherein the heating element (6) is located in the zone of the foot part (2) on the top of the foot part (2).

2. Footwear (1) according to claim 1, wherein the heating element (6) has at least one carrier layer (7) whose length can be changed in one direction, and wherein at least one electrical lead (8) is located in an undulatory manner at least in zones on the carrier layer (7).

3. Footwear (1) according to claim 2, wherein the electrical lead (8) of the heating element (6) is connected to the carrier layer (7), in particular attached and/or cemented on the carrier layer (7), and/or woven and/or stitched on one surface and/or into the carrier layer (7).

4. Footwear (1) according to claim 2, wherein the heating element (6) consists of a supply lead (13) and a heating zone (12).

5. Footwear (1) according to claim 1, wherein the heating element (6) is located on the inside or on the outside of the footwear (1), in particular sewn on or cemented on.

6. Footwear (1) according to claim 4, wherein the heating element (6), in particular the heating zone (12), is located in the toe flap (3).

7. Footwear (1) according to claim 1, wherein a seam, in particular a linking seam (16), for closing the footwear (1) is located between the toe flap (3) and the bottom of the foot part (2).

8. Footwear (1) according to claim 4, wherein the carrier layer (7) of the heating element (6), at least in zones, in particular in a zone (14) of the terminals (9) and in a further zone (15) of the transition from the supply lead (13) to the heating zone (12), has stiffeners that oppose a change in the length of the carrier layer (7).

9. Footwear (1) according to claim 1, wherein the heating element (6) is located from the toe flap (3) over the top of the foot part (2) and then along the shank (4), in particular laterally along, as far as the shank end (5).

10. Footwear (1) according to claim 1, wherein the terminals (9) for the voltage source (11) are located in the zone (14) of the shank end (5), in particular in a cuff zone (10).

11. Footwear (1) according to claim 1, wherein the footwear (1) is made reinforced at least in zones, in particular on the shank (4) and/or on the bottom of the foot part (2).

12. Method for producing footwear (1), in particular a sock, according to claim 1, wherein the heating element (6) is located in the region of the foot part (2) on the top of the foot part (2).

13. Method according to claim 12, wherein the footwear (1) from the shank end (5) to the toe flap (3) is produced, in particular knitted, in the form of a tube, so that an opening remains between the bottom of the foot part (2) and the toe flap (3).

14. Method according to claim 12, wherein the heating element (6) is located on the inside or on the outside of the footwear (1), in particular sewn on or cemented on.

15. Method according to claim 12, wherein the heating element (6), in particular the heating zone (12), is located in the toe flap (3), in particular sewn on or cemented on.

16. Method according to claim 12, wherein the heating element (6) is located, in particular sewn on or cemented on, from the toe flap (3) over the top of the foot part (2) and along the shank (4), in particular laterally along, as far as the shank end (5).

17. Method according to claim 13, wherein the opening between the bottom of the foot part (2) and the toe flap (3) is closed by a seam, in particular a linking seam (16).

18. The footwear of claim 2, wherein the at least one carrier layer is elastic.

19. Footwear (1) according to claim 3, wherein the heating element (6) consists of a supply lead (13) and a heating zone (12).

20. The footwear of claim 4, wherein the electrical lead (8) of the heating element (6) in the zone of the supply lead (13) has a lower ohmic resistance than in the area of the heating zone (12).
